(19) United States
(12) Patent Application Publication
(10) Pub. No.: US 2026/0354934 A1
(43) Pub. Date: Nov. 12, 2026

(54) DISPENSING DEVICE COMPRISING INDEPENDENT DUAL COMPARTMENT AND A DOUBLE CHANNEL TIP

(71) Applicants: António Luís Sardinha Henriques Sales Delgado, Lisboa (PT); EGAS MONIZ – COOPERATIVA DE ENSINO SUPERIOR, Almada (PT)

(72) Inventor: António Luís Sardinha Henriques Sales Delgado, Lisboa (PT)

(21) Appl. No.: 19/142,299

(22) PCT Filed: Dec. 18, 2023
(86) PCT No.: PCT/IB2023/062886

§ 371 Date: Jun. 23, 2025

(30) Foreign Application Priority Data
Dec. 21, 2022 (PT) 118426
Dec. 28, 2022 (EP) 22216874.2

(51) Int. Cl. A61C 5/64

(52) U.S. Cl. CPC A61C 5/64

(57) ABSTRACT

The present disclosure is directed to a device for dispensing at least two different fluids, semi-solids, or a combination thereof, comprising: at least one chamber comprising at least two separate compartments parallel to each other, preferably having different volumes, each compartment with an independent outlet; at least two independent plungers, configured to move independently, and in only a direction towards the independent outlet; a locking mechanism configured to lock at least one plunger, when not being used; and a tip comprising at least two channels, preferably having different areas, wherein each of the channels is in fluid communication with a correspondent independent outlet, and the area of each channel corresponds to an area of an independent outlet. Further systems comprising the device, are further disclosed.

### FIELD OF THE INVENTION

The present disclosure relates generally to the field of dispensers.

### BACKGROUND OF THE INVENTION

Dental composites are mixtures containing heterogenous phases: an organic phase of (meth)acrylate resin monomers dispersed in an inorganic phase comprising filler particles of glass, such as barium or other aluminosilicates, generally bound by an interphase of organosilanes coating the particles. Resin-based dental luting agents have similar composition, except a much lower filler load, or absence of fillers in some formulations. These materials used in restorative dentistry are typically stored in opaque dispensing syringes and from there they can be dispensed to a well or to an auxiliary instrument, or if they are less viscous, they may be directly used in the patient's mouth, when a tooth is being prepared during a restorative procedure.

Recently, formulations of these materials featuring self-adhesive properties have been marketed and commercialized. In the case of self-adhesive luting agents, or in some self-etching dental adhesives, these may contain two-parts that come in separate bottles. This includes a hydrophilic priming coat solution, designated primer, and a more hydrophobic resin mixture as a final coat, which co-polymerize together via light activation. Moreover, self-adhesive dental composites are also flowable resin-based mixtures which have been recently developed and are currently the subject of research focused on their optimization. These formulations are currently stored and dispensed as a one-part syringe system. However, to improve their bonding performance, they may be used with dental adhesives, which come as a separate bottle system, or other simple surface treatments, such as the use of acid, primer alone or novel deproteinizing solutions. Existing dispensing syringes and storage packages do not feature a single system combining two different components, whether they are self-adhesive luting agents or composites with one of the primary surface conditioning/priming steps above mentioned. Also, dental adhesives typically come in bottle systems that cannot be directly dispensed onto the cavity.

Thus, this justifies the need for such invention. Existing syringes do not support dispensing different, independent materials of two-part systems, sequentially, as selected by the operator. Current double-barrel syringes feature a common pushing piston that extrudes both materials at the same time. Furthermore, existing syringes do not contemplate a tip which can extrude two different materials, with different viscosities from independent channels, without mixing.

Patent US20200289237A1 discloses a cartridge and a piston for storing and dispensing a two-component dental material. The cartridge and the piston have a cross-section which substantially correspond to a D-shape in which the perimeter of the D-shape is defined by a plurality of circle segments only.

Patent EP2421464B1 discloses a dispensing device for a dental substance which has an outlet for the dental substance, and a valve for opening and closing the outlet. The device is switchable between a storage mode and an operative mode. In the storage mode a cannula is locked in the device and the valve opens the outlet, and in the storage mode the cannula can be released from the device and the valve closes the outlet.

As such, considering the foregoing, it may be appreciated that there is a need for novel and improved devices containing two independent chambers that may house different, immiscible mixtures, with a single tip that does not mix the components, allowing a sequential dispensing of two separate mixtures.

### SUMMARY OF THE INVENTION

In one aspect of the disclosure, there is provided a device for dispensing at least two different fluids, semi-solids, or a combination thereof, comprising: at least one chamber comprising at least two separate compartments parallel to each other, preferably having different volumes, each compartment with an independent outlet; at least two independent plungers, configured to move independently, towards the independent outlet; a locking mechanism configured to lock at least one plunger, when not being used; and a tip comprising at least two channels, the channels preferably having different areas, wherein each of the channels is in fluid communication with a correspondent independent outlet, and the area of each channel corresponds to an area of an independent outlet.

In a further embodiment, the locking mechanism is further configured to lock at least one plunger from retrieving after being pressed.

In a further embodiment, the locking mechanism comprises a push-button locking mechanism, a locking switch button mechanism, a twist-lock mechanism, a retraction prevention clip, a ring locking mechanism, or any combination thereof

In a further embodiment, the at least two separate compartments are a first and a second compartment, preferably wherein the volume of the first compartment is from 1.5 cm³ to 4 cm³, and the volume of the second compartment is from 6 cm³ to 10 cm³.

In a further embodiment, a diameter of a first outlet is from 0.40 mm to 1.5 mm and a diameter of a second outlet is from 0.80 mm to 3 mm.

In a further embodiment, the tip comprises two independent channels, wherein a diameter of a first independent channel is from 0.40 mm to 1.5 mm and a diameter of a second independent channel is from 0.80 mm to 3 mm, and a total tip diameter is from 1.5 mm to 5 mm.

In a further embodiment, the device further comprises a seal, preferably a polymeric-based seal, a resin-based seal, or a combination thereof, positioned between the at least one chamber and the tip, wherein the seal comprises at least two hoops with different areas, and wherein the area of each hoop corresponds substantially to an area of an independent outlet.

In a further embodiment, the at least two independent plungers comprise slots along at least a portion of its length, positioned from 0.5 mm to³ mm apart, and wherein a length of the slots are configured to dispense an amount from 0.04 cm³ to 0.16 cm³ of the fluids, semi-solids, or a combination thereof.

In a further embodiment, the at least two independent plungers are configured to move a slot in the direction towards the independent outlet, when pressed.

In a further embodiment, the device comprises a button mechanism configured to select a compartment from which to dispense, wherein when selected, the compartment is aligned with a plunger.

In a further embodiment, the tip is metallic, polymeric-based, resin-based, or any combination thereof, and is curved, preferably at an angle from 160° to 110° with relation to the chamber.

In a further embodiment, the at least two different fluids, semi-solids, or a combination thereof, have different viscosities between them.

In a further embodiment, the device is a disposable device, preferably a syringe, a pen-like device, or a multifunctional pen-like device.

In another aspect of the disclosure, there is provided a system comprising a plurality of devices according to the present disclosure.

In another aspect of the disclosure, there is provided a use of the device according to the present disclosure, in dentistry, veterinary medicine, pharmaceutical industry, cosmetic industry, construction industry, laboratory sciences, biomedical engineering, chemical engineering, mechanical engineering, or any combination thereof, preferably for dispensing at least two different fluids, semi-solids, or a combination thereof, without mixing the at least two different fluids, semi-solids, or a combination thereof.

In another aspect of the disclosure, there is provided a use of the system according to the present disclosure, in dentistry, veterinary medicine, pharmaceutical industry, cosmetic industry, construction industry, laboratory sciences, biomedical engineering, chemical engineering, mechanical engineering, or any combination thereof, preferably for dispensing at least two different fluids, semi-solids, or a combination thereof, without mixing the at least two different fluids, semi-solids, or a combination thereof.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

FIG. 1 is an exploded view simplified illustration of some of the components of a device (100) for dispensing at least two fluids, according to some embodiments of the present disclosure;

FIG. 2 is a plan view simplified illustration of some of the components of a device (100) for dispensing at least two fluids, according to some embodiments of the present disclosure;

FIG. 3 is a cross-section view simplified illustration of a chamber (1) and independent outlets (24, 26) of a device (100) for dispensing at least two fluids, according to some embodiments of the present disclosure;

FIG. 4 is a cross-section view simplified illustration of a tip (7), according to some embodiments of the present disclosure;

FIG. 5 is a plan view simplified illustration of a locking mechanism (4), according to some embodiments of the present disclosure;

FIG. 6 is a plan view simplified illustration of a seal (5), according to some embodiments of the present disclosure;

FIG. 7 is a cross-section view simplified illustration of a plunger (2,3), according to some embodiments of the present disclosure;

FIG. 8 is an exploded view simplified illustration of some of the components of a device (200) for dispensing at least two fluids, according to some embodiments of the present disclosure;

FIG. 9 is a is a plan view simplified illustration of some of the components of a device (200) for dispensing at least two fluids, according to some embodiments of the present disclosure;

FIG. 10 is a side view simplified illustration of a device (200), according to some embodiments of the present disclosure; and

FIG. 11 is a is a cross-section view simplified illustration of a device (200), according to some embodiments of the present disclosure.

### DETAILED DESCRIPTION OF THE INVENTION

According to some embodiments, the present disclosure provides a device for dispensing at least two different fluids, semi-solids, or a combination thereof.

As used herein, the term “semi-solid” also encompasses the term “semi-liquid” and refers to any material which is highly viscous, having a thick consistency between a solid and a liquid. According to the present disclosure the term “fluid” refers to a material (such as liquid) that can flow, has no fixed shape, and offers little resistance to an external stress. Non-limiting examples of fluid and semi-solid materials according to the present disclosure include a dental luting agent, a dental adhesive system (comprising a hydrophilic primer mixture and/or a hydrophobic bonding resin), a dental resin composite with varying viscosity, polymeric flowable mixture (e.g., cyanoacrylate glue), a dental irrigant solution, an enzymatic solution, a solvent, an acid for topical use, a micro-emulsion, topical cream, and any combination thereof.

In some embodiments, the device comprises at least one chamber comprising at least two separate compartments parallel to each other and each compartment with an independent outlet. In some embodiments, the device comprises a tip comprising at least two channels, wherein each of the channels is in fluid communication with a correspondent independent outlet. The present disclosure is based, in part, on the finding that a device as described herein may house different, immiscible mixtures, and allows sequential dispensing of the two separate mixtures. In some embodiments, the at least two different fluids, semi-solids, or a combination thereof, have different viscosities.

According to some embodiments, the present disclosure provides a device (100, 200) for dispensing at least two different fluids, semi-solids, or a combination thereof. In some embodiments, the device is a disposable device. In some embodiments, the device is a syringe, a pen-like device, or a multifunctional pen-like device.

The present disclosure is based, in part, on the finding that a tip as described herein comprises at least two channels with different sizes and openings, allowing to accommodate fluids, semi-solids, or a combination thereof, with different viscosities, and also allowing only one mixture to be dispensed at a time.

According to some embodiments, the device comprises at least two independent plungers, configured to move independently, and in only a direction towards the independent outlet. In some embodiments, the device comprises a locking mechanism. The present disclosure is based, in part, on the finding that a device as described herein allows for dispensing only one of the compartments while the other one is locked. In some embodiments, a user of the device chooses at which compartment to dispense from.

It should be understood that according to the present disclosure, a locking mechanism refers to any mechanism able to lock at least one plunger, when not being used. In some embodiments, the locking mechanism is able to lock at least one plunger from retrieving after being pressed. In some embodiments, the locking mechanism can be engaged multiple times when using the device (for several presses of the plunges). Non-limiting examples of suitable locking mechanisms according to the present disclosure include: i) push-button locking mechanism, where a button is pressed to move one of the plungers being used and the button locks or engages, preventing the plunger from being pulled back and/or a second plunger to move; ii) locking switch button mechanism, where after pressed, the button is engaged with a switch; iii) twist-lock mechanism, where the plunger being used twists or locks into place after it's been fully depressed; iv) retraction prevention clips or ring locking mechanism, where an additional clip, ring or a component prevents the plunger from being pulled back once it's been fully depressed and/or a second plunger from moving.

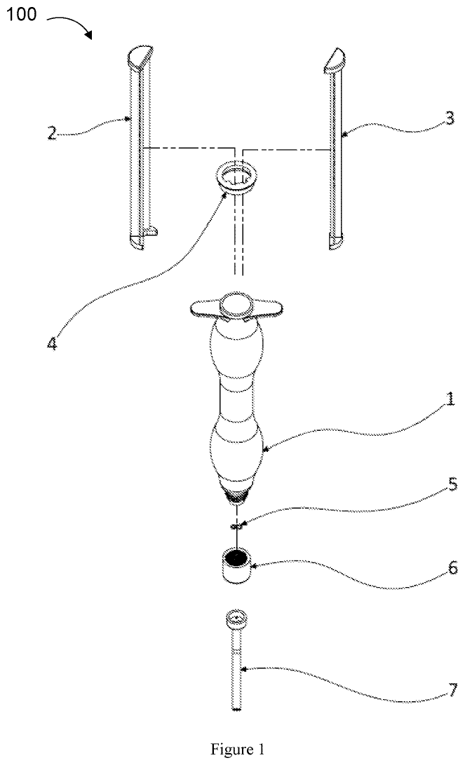

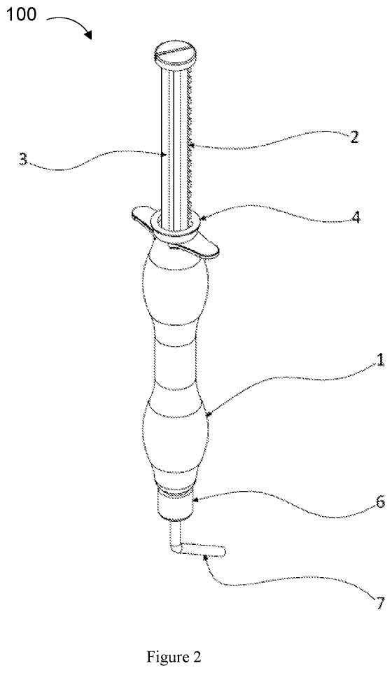

Reference is made to FIG. 1 and FIG. 2 which are an exploded view and a plan view simplified illustration of some of the components of a device (100) for dispensing at least two fluids, according to some embodiments of the present disclosure.

According to some embodiments, a device (100) according to the present disclosure comprises at least one chamber (1) comprising at least two separate compartments parallel to each other and each compartment with an independent outlet. The device comprises at least two independent plungers (2, 3), configured to move independently, and in only a direction towards the independent outlet. The device comprises a locking mechanism (4) and a tip (7) comprising at least two channels, wherein each of the channels is in fluid communication with a correspondent independent outlet.

In some embodiments, the device (100) comprises a seal (5) positioned between the at least one chamber and the tip. In some embodiments, the seal (5) is a polymeric-based seal, a resin-based seal, or a combination thereof. In some embodiments, the seal comprises at least two hoops with different areas, wherein the area of each hoop corresponds substantially to an area of an independent outlet. According to the present disclosure, a seal as described herein prevents the liquid of a material (fluids, semi-solids, or a combination thereof) from the compartments to outside of the chamber.

In some embodiments, the device (100) comprises a screw mechanism (6) configured to connect the at least one chamber (1) and the tip (7). In some embodiments, the tip (7) connects directly to the at least one chamber (1). Means to connect a chamber and a tip are well known in the art and will become apparent to a person skillet in the art. Non-limiting examples of means to connect a chamber (1) and a tip (7) include screw-type connection, click-type connection, complementary joint connections.

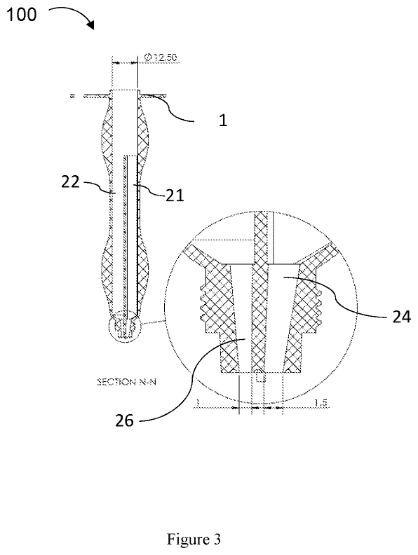

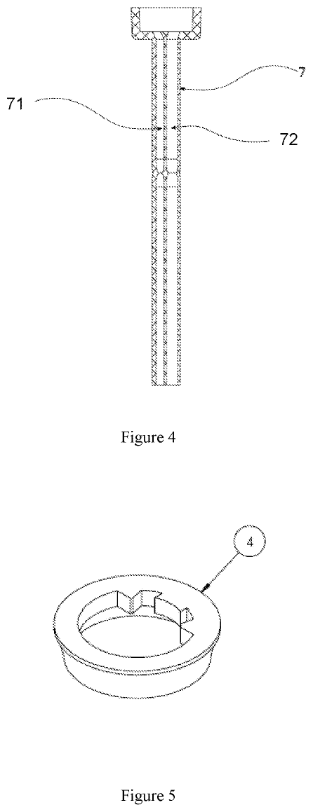

Reference is now made to FIG. 3 which represents a cross-section view simplified illustration of a chamber (1) and independent outlets (24, 26) of a device (100), and FIG. 4 which represents a cross-section view simplified illustration of a tip (7), according to some embodiments of the present disclosure.

According to some embodiments, a at least one chamber (1) comprises at least two separate compartments (21, 22). In some embodiments, the at least two separate compartments have different volumes.

According to some embodiments, a at least one chamber (1) comprises two separate compartments (21, 22), wherein a volume of a first compartment (21 or 22) is between 1.5 cm³ and 4 cm³, and a volume of a second compartment (22 if the first compartment is 21 or 21 if the first compartment is 22) is between 6 cm³ and 10 cm^{3.}

In some embodiments, a volume of a first compartment is between 1.5 cm³ and 4 cm³, between 1.8 cm³ and 4 cm³, between 2 cm 3 and 4 cm³, between 2.5 cm³ and 4 cm³, between 3 cm³ and 4 cm³, between 1.5 cm³ and 3 cm³, between 1.8 cm³ and 3 cm³, between 2 cm³ and 3 cm³, between 2.5 cm³ and 3 cm³, between 1.5 cm³ and 2 cm³, or between 1.8 cm³ and 2 cm³, including any range therebetween. Each possibility represents a separate embodiment of the present disclosure.

In some embodiments, a volume of a second compartment is between 6 cm³ and 10 cm³, between 6.5 cm³ and 10 cm³, between 7 cm³ and 10 cm³, between 8 cm³ and 10 cm³, between 8.5 cm³ and 10 cm³, between 9 cm³ and 10 cm³, between 6 cm³ and 9.5cm³, between 6.5 cm³ and 9.5 cm³, between 7 cm³ and 9.5cm³, between 8 cm³ and 9.5cm³, or between 8.5 cm³ and 9.5cm³, including any range therebetween. Each possibility represents a separate embodiment of the present disclosure.

The present disclosure is based, in part, on the finding that a volume of each of the at least two compartments is determined according to the chosen fluids, semi-solids, or a combination thereof and according to their viscosity and density, allowing for an accurate and waste-minimizing dispensing of the desired amount of each fluid, semi-solid, or a combination thereof.

As represented in FIG. 3, each of the at least two compartments (21, 22) comprise a corresponding independent outlet (24, 26). In some embodiments, each independent outlet (24, 26) has a different diameter.

In some embodiments, a device (100) comprises two independent outlets (24, 26), wherein a diameter of a first outlet (24 or 26) is between 0.40 mm and 1.5 mm and a diameter of a second outlet (26 if the first outlet is 24 or 24 if the first outlet is 26) is between 0.80 mm and 3 mm.

In some embodiments, a diameter of a first outlet is between 0.40 mm and 1.5 mm, between 0.45 mm and 1.5 mm, between 0.5 mm and 1.5 mm, between 0.7 mm and 1.5 mm, between 0.9 mm and 1.5 mm, between 0.40 mm and 1 mm, between 0.45 mm and 1 mm, between 0.5 mm and 1 mm, between 0.7 mm and 1 mm, or between 0.9 mm and 1 mm, including any range therebetween. Each possibility represents a separate embodiment of the present disclosure.

In some embodiments, a diameter of a second outlet is between 0.80 mm and 3 mm, between 0.90 mm and 3 mm, between 1 mm and 3 mm, between 1.5 mm and 3 mm, between 2 mm and 3 mm, between 0.80 mm and 2.5 mm, between 0.90 mm and 2.5 mm, between 1 mm and 2.5 mm, between 1.5 mm and 2.5 mm, between 2 mm and 2.5 mm, between 0.80 mm and 2 mm, between 0.90 mm and 2 mm, between 1 mm and 2 mm, or between 1.5 mm and 2 mm, including any range therebetween. Each possibility represents a separate embodiment of the present disclosure.

In some embodiments, the at least two independent outlets arc tapered towards their exit. The present disclosure is based in part, on the finding that the taper allows better control of material flow (fluid, semi-solid, or a combination thereof), and less quantity is wasted with each pressing of the device since there is resistance in the outflow due to convergence in the exit.

As represented in FIG. 4, a tip (7) comprises two different channels (71, 72). In some embodiments, the at least two channels (71, 72) have different areas, wherein the area of each channel (71, 72) corresponds to an area of an independent outlet (24, 26).

In some embodiments, the tip (7) comprises two independent channels (71,72) wherein a diameter of a first independent channel (71 or 72) is between 0.40 mm and 1.5 mm and a diameter of a second independent channel (72 if the first channel is 71 or 71 if the first channel is 72) is between 0.80 mm and 3 mm.

In some embodiments, a diameter of a first channel is between 0.40 mm and 1.5 mm, between 0.45 mm and 1.5 mm, between 0.5 mm and 1.5 mm, between 0.7 mm and 1.5 mm, between 0.9 mm and 1.5 mm, between 0.40 mm and 1 mm, between 0.45 mm and 1 mm, between 0.5 mm and 1 mm, between 0.7 mm and 1 mm, or between 0.9 mm and 1 mm, including any range therebetween. Each possibility represents a separate embodiment of the present disclosure.

In some embodiments, a diameter of a second channel is between 0.80 mm and 3 mm, between 0.90 mm and 3 mm, between 1 mm and 3 mm, between 1.5 mm and 3 mm, between 2 mm and 3 mm, between 0.80 mm and 2.5 mm, between 0.90 mm and 2.5 mm, between 1 mm and 2.5 mm, between 1.5 mm and 2.5 mm, between 2 mm and 2.5 mm, between 0.80 mm and 2 mm, between 0.90 mm and 2 mm, between 1 mm and 2 mm, or between 1.5 mm and 2 mm, including any range therebetween. Each possibility represents a separate embodiment of the present disclosure.

In some embodiments, a total tip diameter is between 1.5 mm to 5 mm, between 1.9 mm to 5 mm, between 2 mm to 5 mm, between 2.5 mm to 5 mm, between 3 mm to 5 mm, between 4 mm to 5 mm, between 1.5 mm to 4.5 mm, between 1.9 mm to 4.5 mm, between 2 mm to 4.5 mm, between 2.5 mm to 4.5 mm, between 3 mm to 4.5 mm, between 1.5 mm to 4 mm, between 1.9 mm to 4 mm, between 2 mm to 4 mm, between 2.5 mm to 4 mm, or between 3 mm to 4 mm, including any range therebetween. Each possibility represents a separate embodiment of the present disclosure.

In some embodiments, the tip (7) is metallic, polymeric-based, resin-based, or any combination thereof. In some embodiments, the tip (7) is curved, preferably at an angle between 160° and 110° with relation to the chamber. In some embodiments, the tip (7) is curved and fixed at the described angle. In some embodiments, the tip is flexible and can move between an angle orientation as described, in only a plane (e.g., in the Y plane). The present disclosure is based, in part, on the finding that a device as described herein with a flexible tip (with an adjustable angle) can be directed inside e.g., an oral cavity, to a chosen and needed angle.

Reference is now made to FIG. 5, which represents a locking mechanism (4) according to some embodiments of the present disclosure. In some embodiments, the locking mechanism (4) is a locking ring. In some embodiments, the locking mechanism is configured to lock at least one plunger, when not being used. The mechanism functions through macromechanical interlocking, where the locking mechanism engages the plunger not in use, latching its slots, thus preventing it from moving downwards.

In some embodiments, the locking mechanism is configured to lock at least one plunger from retrieving after being pressed. The mechanism functions through micromechanical interlocking, where the locking mechanism engages the plunger, latching its slots, thus preventing it from moving upwards.

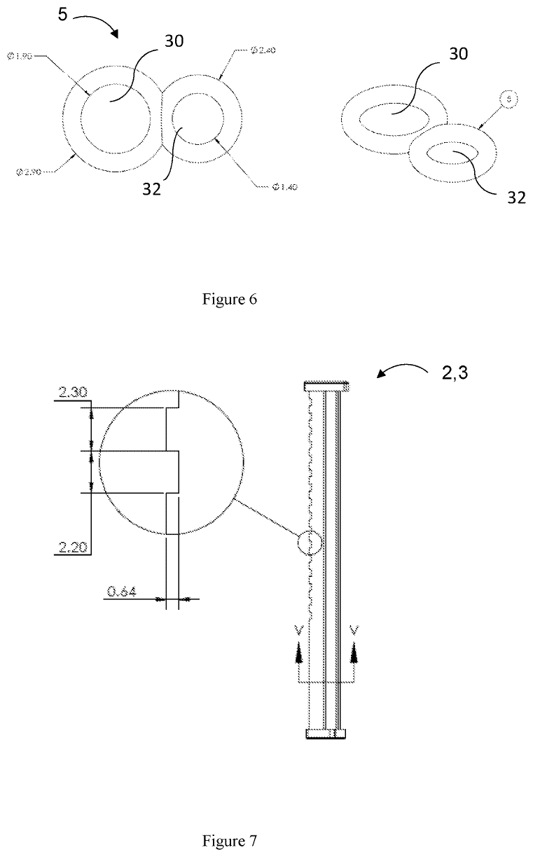

Reference is now made to FIG. 6, which represents a seal (5) according to some embodiments of the present disclosure.

In some embodiments, the device (100) comprises a seal (5) positioned between the at least one chamber and the tip. In some embodiments, the seal (5) comprises at least two hoops (30, 32) with different areas, wherein the area of each hoop corresponds substantially to an area of an independent outlet.

In some embodiments, the seal (5) comprises two hoops (30, 32). In some embodiments, a diameter of a first hoop is between 0.40 mm and 1.5 mm, between 0.45 mm and 1.5 mm, between 0.5 mm and 1.5 mm, between 0.7 mm and 1.5 mm, between 0.9 mm and 1.5 mm, between 0.40 mm and 1 mm, between 0.45 mm and 1 mm, between 0.5 mm and 1 mm, between 0.7 mm and 1 mm, or between 0.9 mm and 1 mm, including any range therebetween. Each possibility represents a separate embodiment of the present disclosure. In some embodiments, a diameter of a second hoop is between 0.80 mm and 3 mm, between 0.90 mm and 3 mm, between 1 mm and 3 mm, between 1.5 mm and 3 mm, between 2 mm and 3 mm, between 0.80 mm and 2.5 mm, between 0.90 mm and 2.5 mm, between 1 mm and 2.5 mm, between 1.5 mm and 2.5 mm, between 2 mm and 2.5 mm, between 0.80 mm and 2 mm, between 0.90 mm and 2 mm, between 1 mm and 2 mm, or between 1.5 mm and 2 mm, including any range therebetween. Each possibility represents a separate embodiment of the present disclosure.

In some embodiments, the at least two hoops (30, 32) have a thickness between, 0.4 and 2 mm, between 0.5 mm and 2 mm, between 0.7 mm and 2 mm, between 0.9 mm and 2 mm, between 0.40 mm and 1.5 mm, between 0.45 mm and 1.5 mm, between 0.5 mm and 1.5 mm, between 0.7 mm and 1.5 mm, between 0.9 mm and 1.5 mm, between 0.40 mm and 1 mm, between 0.45 mm and 1 mm, between 0.5 mm and 1 mm, between 0.7 mm and 1 mm, or between 0.9 mm and 1 mm, including any range therebetween. Each possibility represents a separate embodiment of the present disclosure.

Reference is now made to FIG. 7, which represents one of the at least two independent plungers (2, 3), according to some embodiments of the present disclosure.

In some embodiments, the at least two independent plungers (2, 3) comprise slots along at least a portion of its length, as represented in FIG. 7, positioned between 0.5 mm and 3 mm apart, between 1 mm and 3 mm apart, between 1.5 mm and 3 mm apart, between 2 mm and 3 mm apart, between 0.5 mm and 2.9 mm apart, between 1 mm and 2.9 mm apart, between 1.5 mm and 2.9 mm apart, between 2 mm and 2.9 mm apart, between 0.5 mm and 2.5 mm apart, between 1 mm and 2.5 mm apart, between 1.5 mm and 2.5 mm apart, or between 2 mm and 2.5 mm apart, including any range therebetween. Each possibility represents a separate embodiment of the present disclosure.

In some embodiments, a length of the slots is adjusted to dispense an amount between 0.04 cm³ and 0.16 cm³ of the fluids, semi-solids, or a combination thereof.

In some embodiments, the at least two independent plungers (2, 3) are configured to move a slot in the direction towards the independent outlet, when pressed.

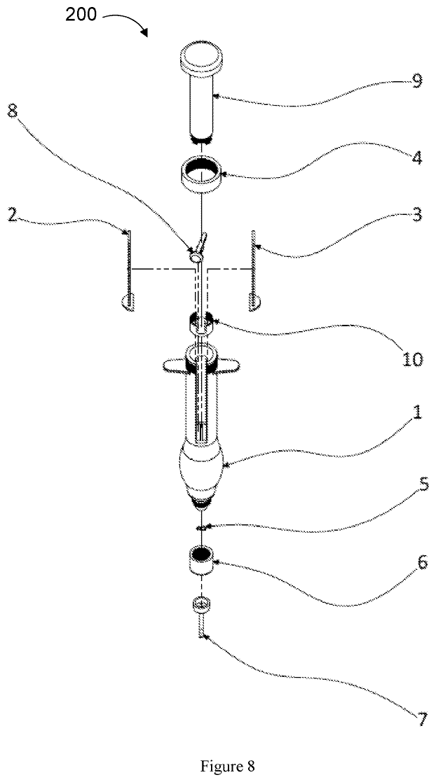

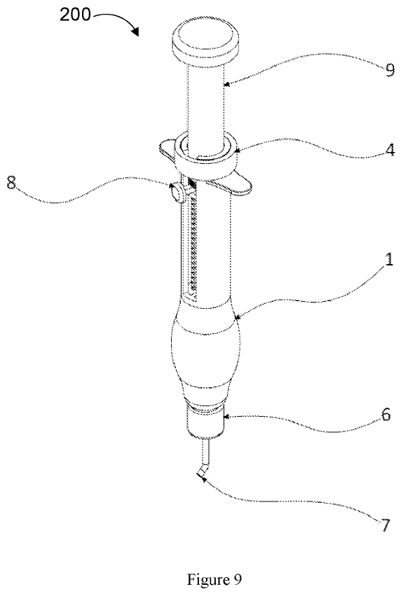

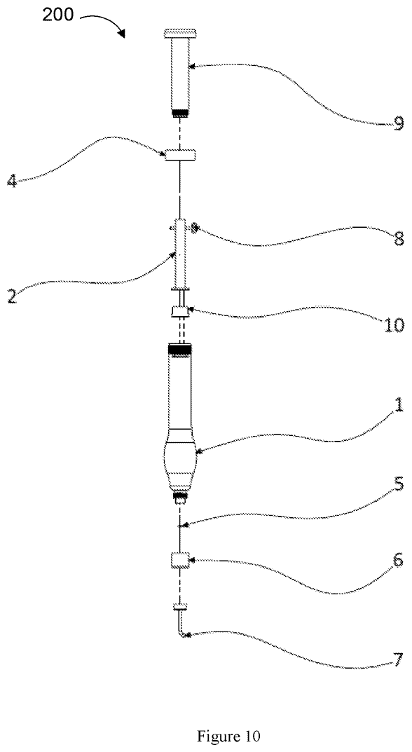

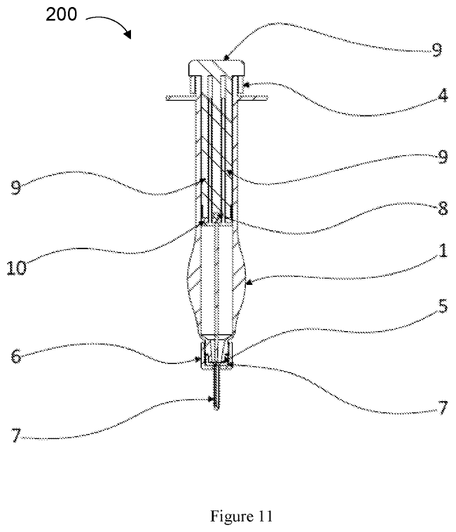

Reference is now made to FIG. 8, which is an exploded view simplified illustration of some of the components of a device (200) for dispensing at least two fluids, according to some embodiments of the present disclosure.

According to some embodiments of the present disclosure, a device as described herein comprises a button mechanism, configured to select a compartment from which to dispense, wherein when selected, the compartment is aligned with a plunger. In some embodiments, a device (200) comprises a button mechanism, configured to select a compartment from which to dispense, wherein when selected, the compartment is aligned with a plunger.

In some embodiments, the button mechanism comprises a push rod and an engagement knob.

In some embodiments, a device (200) comprises a push rod (9) and an engagement knob (8), wherein the engagement knob (8) is in contact with the push rod (9) and is configured to select one of the at least two independent plungers (2, 3), allowing the push rod to actuate only in the independent plunger chosen, while the other plunger is locked. If plungers are at different dispensing positions (such as in cases where it is possible to empty one of the chambers first), it may be required to take the main push rod back, so the selector button can catch the opposite side.

In some embodiments, a device (200) further comprises an additional padlock (10) on push rod (9) that prevents the two independent plungers (2, 3) from coming out of the chamber (1). In some embodiments, the padlock (10) is in contact with the fluids, semi-solids, or a combination thereof, present in each compartment.

In some embodiments, the engagement knob (8), acts like a selector button allowing to select the plunger on the left or right, so that the push rod (9) acts like a main piston that descends and acts on it. The center of the engagement knob (8) is fixed to the push rod (9). In some embodiments, a mechanism as described herein works in “swipe”-like motion. In some embodiments, a user can swipe left or right.

According to some embodiments, the present disclosure, provides a system comprising a plurality of devices as described hereinabove.

According to some embodiments, the present disclosure, provides a use of a device as described hereinabove, for dispensing at least two different fluids, semi-solids, or a combination thereof, without mixing the at least two different fluids, semi-solids, or a combination thereof.

According to some embodiments, the present disclosure, provides a use of a device as described hereinabove, in dentistry, veterinary medicine, pharmaceutical industry, cosmetic industry, construction industry, laboratory sciences, biomedical engineering, chemical engineering, mechanical engineering, or any combination thereof.

The terms “comprises”, “comprising”, “includes”, “including”, “having” and their conjugates mean “including but not limited to”.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. Device for dispensing at least two different fluids, semi-solids, or a combination thereof, comprising:
at least one chamber comprising at least two separate compartments parallel to each other, preferably having different volumes, each compartment with an independent outlet;
at least two independent plungers, configured to move independently towards said independent outlet;
a locking mechanism configured to lock at least one plunger, when not being used; and
a tip comprising at least two channels, the channels preferably having different areas, wherein each of said channels is in fluid communication with a correspondent independent outlet, and the area of each channel corresponds to an area of an independent outlet.

2. Device according to claim 1, wherein said locking mechanism is further configured to lock at least one plunger from retrieving after being pressed.

3. Device according to any one of claims 1 to 2, wherein said locking mechanism comprises a push-button locking mechanism, a locking switch button mechanism, a twist-lock mechanism, a retraction prevention clip, a ring locking mechanism, or any combination thereof.

4. Device according to claim 1 or 3, wherein the at least two separate compartments are a first and a second compartment, preferably wherein the volume of the first compartment is from 1.5 cm³ to 4 cm³, and the volume of the second compartment is from 6 cm³ to 10 cm^{3.}

5. Device according to claim 4, wherein a diameter of a first outlet is from 0.40 mm to 1.5 mm and a diameter of a second outlet is from 0.80 mm to 3 mm.

6. Device according to any one of claims 4 to 5, wherein said tip comprises two independent channels, wherein the diameter of a first independent channel is from 0.40 mm to 1.5 mm and a diameter of a second independent channel is from 0.80 mm to 3 mm, and a total tip diameter is from 1.5 mm to 5 mm.

7. Device according to any one of claims 1 to 6, further comprising a seal, preferably a polymeric-based seal, a resin-based seal, or a combination thereof, positioned between said at least one chamber and said tip, wherein said seal comprises at least two hoops with different areas, and wherein the area of each hoop corresponds substantially to an area of an independent outlet.

8. Device according to any one of claims 1 to 7, wherein said at least two independent plungers comprise slots along at least a portion of its length, positioned from 0.5 mm to 3 mm apart, and wherein a length of said slots are configured to dispense an amount from 0.04 cm³ to 0.16 cm³ of said fluids, semi-solids, or a combination thereof.

9. Device according to claim 8, wherein said at least two independent plungers are configured to move a slot in the direction towards said independent outlet, when pressed.

10. Device according to any one of claims 1 to 9, comprising a button mechanism configured to select a compartment from which to dispense, wherein when selected, said compartment is aligned with a plunger.

11. Device according to any one of claims 1 to 10, wherein said tip is metallic, polymeric-based, resin-based, or any combination thereof, and is curved, preferably at an angle from 160° to 110° with relation to said chamber.

12. Device according to any one of claims 1 to 11, wherein said at least two different fluids, semi-solids, or a combination thereof, have different viscosities between them.

13. Device according to any one of claims 1 to 12, wherein said device is a disposable device, preferably a syringe, a pen-like device, or a multifunctional pen-like device.

14. System comprising a plurality of devices according to any one of claims 1 to 13.

15. Use of the device according to any one of claims 1 to 13, in dentistry, veterinary medicine, pharmaceutical industry, cosmetic industry, construction industry, laboratory sciences, biomedical engineering, chemical engineering, mechanical engineering, or any combination thereof, preferably for dispensing at least two different fluids, semi-solids, or a combination thereof, without mixing said at least two different fluids, semi-solids, or a combination thereof.

16. Use of the system according to claim 14, in dentistry, veterinary medicine, pharmaceutical industry, cosmetic industry, construction industry, laboratory sciences, biomedical engineering, chemical engineering, mechanical engineering, or any combination thereof, preferably for dispensing at least two different fluids, semi-solids, or a combination thereof, without mixing said at least two different fluids, semi-solids, or a combination thereof.
